(19) United States
(12) Patent Application Publication
(10) Pub. No.: US 2020/0376247 A1
(43) Pub. Date: Dec. 3, 2020

(54) METHOD OF PRODUCING MICRONEEDLE ARRAY

(71) Applicant: FUJIFILM Corporation, Tokyo (JP)

(72) Inventors: Toshio SHIMADA, Ashigarakami-gun (JP); Koki KABATA, Ashigarakami-gun (JP)

(73) Assignee: FUJIFILM Corporation, Tokyo (JP)

(21) Appl. No.: 16/997,696

(22) Filed: Aug. 19, 2020

Related U.S. Application Data
(63) Continuation of application No. PCT/JP2019/006230, filed on Feb. 20, 2019.

(30) Foreign Application Priority Data
Feb. 20, 2018 (JP) 2018-027824

(51) Int. Cl. A61M 37/00; A61K 9/00

(52) U.S. Cl. CPC A61M 37/0015; A61K 9/0021; A61M 2037/0023; A61M 2037/0053; A61M 2037/0061; A61M 2037/0046

(57) ABSTRACT

An object of the present invention is to provide a method of producing a microneedle array that enables concentration of a drug at needle tips of the microneedle array. According to the present invention, provided is a method of producing a microneedle array, including a step of filling a hydrophobic mold with a drug-containing solution to form a needle tip, and a step of filling the mold including the formed needle tip with a liquid which contains a water-soluble polymer or disaccharides to form a needle base and a sheet, in which the drug-containing solution contains 0.01 mg/mL to 5 mg/mL of a surfactant.

### CROSS-REFERENCE TO RELATED APPLICATIONS

This application is a Continuation of PCT International Application No. PCT/JP2019/006230 filed on Feb. 20, 2019, which claims priority under 35 U.S.C § 119(a) to Japanese Patent Application No. 2018-027824 filed on Feb. 20, 2018. Each of the above application(s) is hereby expressly incorporated by reference, in its entirety, into the present application.

### BACKGROUND OF THE INVENTION

### 1. Field of the Invention

The present invention relates to a method of producing a microneedle array and particularly a method of producing a self-dissolving microneedle array containing a drug.

### 2. Description of the Related Art

As a method of administering a drug for administering an appropriate amount of a drug to achieve sufficient drug efficacy, a microneedle array of allowing microneedles to penetrate a keratin barrier layer using a microneedle array in which microneedles (needles) containing a drug and having a high aspect ratio have been formed and injecting the drug into the skin without pain has been attracting attention. For example, a self-dissolving microneedle array using a substance having solubility in vivo as a base material has been reported. In the self-dissolving microneedle array, a drug can be administered intradermally by allowing the base material to hold the drug so that self-dissolution of the base material occurs in a case of inserting microneedles into the skin.

WO2014/077244A describes a method of producing a transdermal absorption sheet, including a laminating step of forming an underlayer that contains a first transdermal absorption material and an upper layer that contains a drug and a second transdermal absorption material and has a lower viscosity than that of the underlayer, to form a multi-layer film with a difference in viscosity; a filling step of pressing a mold, in which needle-like recesses formed by needle-like projections being inverted have been two-dimensionally arranged, against a surface of the multi-layer film supported by a support so that the multi-layer film flows and filling the needle-like recesses with a solution of the transdermal absorption material; a solidifying step of solidifying the multi-layer film in a state in which the mold is pressed against the surface of the multi-layer film; and a peeling step of peeling the solidified multi-layer film off from the mold.

### SUMMARY OF THE INVENTION

In order to product a self-dissolving microneedle array that contains a drug, it is necessary to mix the drug into the microneedle array. However, since the drug is mostly expensive, the drug needs to be concentrated at a needle tip. As a method of allowing the drug to be concentrated at the needle tip, a method of performing multi-stage filling of a mold that contains a hydrophobic material as described in WO2014/077244A is known. Meanwhile, since most drugs are likely to be adsorbed on a surface of a hydrophobic material, the concentration of a drug at a needle tip is disturbed in many cases.

An object of the present invention is to provide a method of producing a microneedle array that enables concentration of a drug at needle tips of a microneedle array.

As a result of intensive examination conducted by the present inventors in order to achieve the above-described object, it was found that a microneedle array that enables concentration of a drug at needle tips of a microneedle array can be produced by allowing a drug-containing solution to contain 0.01 mg/mL to 5 mg/mL of a surfactant using a method of producing a microneedle array, including a step of filling a hydrophobic mold with a drug-containing solution to form needle tips; and a step of filling the mold that includes the formed needle tips with a liquid containing a water-soluble polymer or disaccharides to form a needle base and a sheet. The present invention has been completed based on these findings.

That is, according to the present invention, the following inventions are provided.

(1) A method of producing a microneedle array, comprising: a step of filling a hydrophobic mold with a drug-containing solution to form a needle tip; and a step of filling the mold including the formed needle tip with a liquid which contains a water-soluble polymer or disaccharides to form a needle base and a sheet, in which the drug-containing solution contains 0.01 mg/mL to 5 mg/mL of a surfactant.

(2) The method according to (1), in which the surfactant is a nonionic surfactant.

(3) The method according to (1) or (2), in which a mass of the drug in a needle tip region including the needle tip and having a height corresponding to a length of 2/3 or 575/800 of a height of an entire needle is 80% or greater of a total mass of the drug that has filled the mold.

(4) The method according to any one of (1) to (3), in which the drug includes a peptide or a vaccine.

(5) The method according to any one of (1) to (4), in which the mold has a silicon atom or a carbon atom.

According to the present invention, it is possible to produce a microneedle array which enables concentration of a drug at needle tips of the microneedle array.

### BRIEF DESCRIPTION OF THE DRAWINGS

FIG. 1A is a perspective view illustrating a conical microneedle, FIG. 1B is a perspective view illustrating a pyramid-like microneedle, and FIG. 1C is a cross-sectional view illustrating a conical and pyramid-like microneedle.

FIG. 2 is a perspective view illustrating a microneedle in another shape.

FIG. 3 is a perspective view illustrating a microneedle in another shape.

FIG. 4 is a cross-sectional view of the microneedles illustrated in FIGS. 2 and 3.

FIG. 5 is a perspective view illustrating a microneedle in another shape.

FIG. 6 is a perspective view illustrating a microneedle in another shape.

FIG. 7 is a cross-sectional view of the microneedles illustrated in FIGS. 5 and 6.

FIG. 8 is a cross-sectional view of a microneedle in another shape in which the inclination (angle) of a side surface of a needle is continuously changed.

FIGS. 9A to 9C are step views illustrating a method of producing a mold.

FIG. 10 is an enlarged view of a mold.

FIG. 11 is a cross-sectional view illustrating a mold in another shape.

FIGS. 12A to 12C are schematic views illustrating a step of filling a mold with a drug-containing solution.

FIG. 13 is a perspective view illustrating an end of a nozzle.

FIG. 14 is a partially enlarged view illustrating the end of the nozzle and the mold during the filling.

FIG. 15 is a partially enlarged view illustrating the end of the nozzle and the mold during movement.

FIGS. 16A to 16D are views for describing a step of forming another microneedle array.

FIGS. 17A to 17C are views for describing a step of forming still another microneedle array.

FIG. 18 is a view for describing a peeling step.

FIG. 19 is a view for describing another peeling step.

FIG. 20 is a view for describing a microneedle array.

FIGS. 21A and 21B are respectively a plan view and a side view of an original plate.

### DESCRIPTION OF THE PREFERRED EMBODIMENTS

Hereinafter, embodiments of the present invention will be described in detail.

In the present specification, the expression “containing a drug” means that a drug having an amount enough to exhibit drug efficacy is contained in a case of puncturing the body surface. The expression “not containing a drug” means that a drug having an amount enough to exhibit drug efficacy is not contained, and a range of the amount of the drug covers from a case where the drug is not contained at all to a case where the amount thereof is not enough to exhibit the drug efficacy.

[Configuration of Microneedle Array]

A microneedle array to be produced according to a method according to the embodiment of the present invention is a microneedle array including a sheet and a plurality of needles that are present on an upper surface of the sheet.

In the present invention, plural means one or more.

The microneedle array includes at least a sheet and needles and a drug is carried and supported by the needles in order to efficiently administer the drug into the skin.

The microneedle array is a device in which a plurality of needles are arranged in an array on the upper surface side of the sheet. It is preferable that the needles are arranged on the upper surface side of the sheet. The needles may be arranged directly on the upper surface of the sheet or may be arranged on the upper surfaces of frustums arranged on the upper surface of the sheet.

It is preferable that the needles are arranged on the upper surfaces of frustums arranged on the upper surface of the sheet. In this case, the microneedle array of the present invention includes a plurality of frustums between the sheet and a plurality of needles. According to the embodiment, it is preferable a needle tip contains a drug, a surfactant, and at least one of a water-soluble polymer or disaccharides, and a needle base, a frustum, and a sheet contain at least one of a water-soluble polymer or disaccharides. The water-soluble polymer contained in the needle and the sheet may be the same as or different from each other. The water-soluble polymer will be described below.

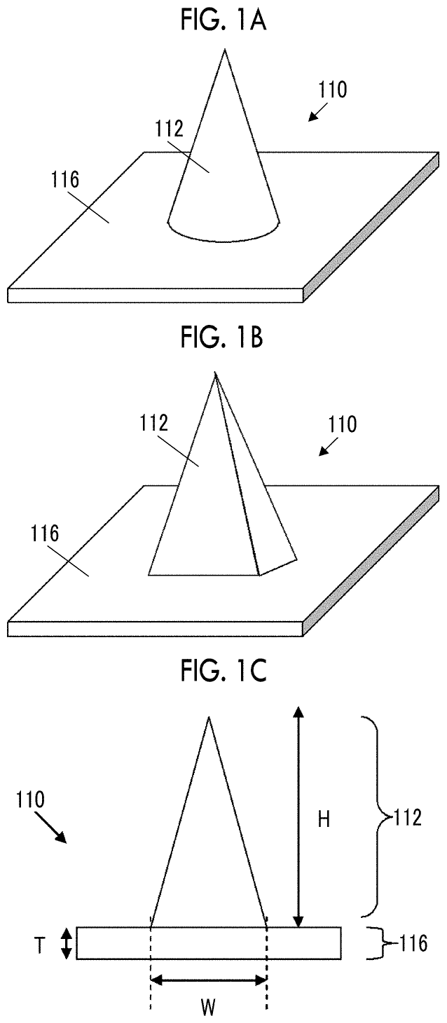

The sheet is a foundation for supporting needles and has a planar shape as the shape of the sheet 116 illustrated in FIGS. 1 to 8. In this case, the upper surface of the sheet indicates the surface on which the plurality of needles are arranged in an array.

The area of the sheet is not particularly limited, but is preferably in a range of 0.005 to 1000 mm², more preferably in a range of 0.05 to 500 mm², and still more preferably in a range of 0.1 to 400 mm².

The thickness of the sheet is a distance between the surface in contact with frustums or needles and the surface on the opposite side. The thickness of the sheet is preferably in a range of 1 μm to 2000 μm, more preferably in a range of 3 μm to 1500 μm, and still more preferably in a range of 5 μm to 1000 μm.

The sheet contains at least one of a water-soluble polymer or disaccharides. The sheet may contain additives other than the water-soluble polymer and the disaccharides. Further, it is preferable that the sheet does not contain a drug.

The water-soluble polymer contained in the sheet is not particularly limited, and examples thereof include polysaccharides, polyvinylpyrrolidone, polyoxyethylene polyoxypropylene glycol, polyethylene glycol, polyvinyl alcohol, and protein (for example, gelatin). Examples of the polysaccharides include hyaluronic acid, sodium hyaluronate, pullulan, dextran, dextrin, chondroitin sulfate, sodium chondroitin sulfate, a cellulose derivative (for example, a water-soluble cellulose derivative obtained by partially modifying cellulose such as carboxymethyl cellulose, hydroxypropyl cellulose, or hydroxypropyl methylcellulose), hydroxyethyl starch, and gum arabic. The above-described components may be used alone or in the form of a mixture of two or more kinds thereof.

Among these, as the water-soluble polymer contained in the sheet, at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol is preferable, and chondroitin sulfate is particularly preferable.

The weight-average molecular weight of the water-soluble polymer contained in the sheet is preferably in a range of 5000 to 200000, more preferably in a range of 10000 to 150000, and still more preferably in a range of 30000 to 120000.

Disaccharides may be added to the sheet, and examples of the disaccharides include sucrose, lactulose, lactose, maltose, trehalose, and cellobiose. Among these, sucrose, maltose, and trehalose are particularly preferable.

The microneedle array is formed of a plurality of needles arranged in an array on the upper surface side of the sheet. The needles have a projected structure with a tip, and the shape thereof is not limited to a needle shape having a sharp tip and may be a shape with a blunt tip.

Examples of the shape of a needle include a conical shape, a polygonal pyramid shape (square pyramid shape or the like), and a spindle shape. For example, a needle may have a shape of a needle 112 illustrated in FIGS. 1 to 8, in which the entire shape of the needle may be a conical shape, a polygonal pyramid shape (square pyramid shape or the like), or a shape of a structure in which the inclination (angle) of the side surface of the needle is continuously changed. Further, a needle may have a multilayer structure with two or more layers, in which the inclination (angle) of the side surface of the needle is discontinuously changed.

In a case where the microneedle array of the present invention is applied to the skin, it is preferable that the needles are inserted into the skin and the upper surface or a part of the sheet is brought into contact with the skin.

The height (length) of a needle indicates the length of a perpendicular line drawn from the tip of the needle to a frustum or the sheet (in a case where a frustum is not present). The height (length) of a needle is not particularly limited, but is preferably in a range of 50 μm to 3000 μm, more preferably in a range of 100 μm to 1500 μm, and still more preferably in a range of 100 μm to 1000 μm. It is preferable that the length of a needle is 50 μm or greater because a drug can be percutaneously administered. Further, it is preferable that the length of a needle is 3000 μm or less because occurrence of pain resulting from the contact of needles with the nerve is prevented and bleeding can be avoided.

The interface between a frustum (or a needle in a case where a frustum is not present) and the sheet is referred to as a base portion. The distance between a base of one needle and a point farthest from the base is preferably in a range of 50 μm to 2000 μm, more preferably in a range of 100 μm to 1500 μm, and still more preferably in a range of 200 μm to 1000 μm.

The number of needles to be arranged in one microneedle array is preferably in a range of 1 to 2000, more preferably in a range of 3 to 1000, and still more preferably in a range of 5 to 500. In a case where one microneedle array includes two needles, the interval between needles indicates the distance between feet of each perpendicular line drawn from the tip of a needle to a frustum or the sheet (in the case where a frustum is not present). In a case where one microneedle array includes three or more needles, the interval between needles to be arranged indicates an average value obtained by acquiring the distance between a foot of a perpendicular line drawn from the tip of a needle to a frustum or the sheet (in the case where frustums are not present) and a foot of a perpendicular line drawn from the tip of a needle nearest to the needle to a frustum or the sheet and averaging the values obtained from all needles. The interval between needles is preferably in a range of 0.1 mm to 10 mm, more preferably in a range of 0.2 mm to 5 mm, and still more preferably in a range of 0.3 mm to 3 mm.

A needle contains a drug, a surfactant, and at least one of a water-soluble polymer or disaccharides.

It is preferable that the water-soluble polymer is a biosoluble substance such that a human body is not damaged even in a case where needles remain in the skin.

The water-soluble polymer contained in the needles is not particularly limited, and examples thereof include polysaccharides, polyvinylpyrrolidone, polyoxyethylene polyoxypropylene glycol, polyethylene glycol, polyvinyl alcohol, and protein (for example, gelatin). Examples of the polysaccharides include hyaluronic acid, sodium hyaluronate, pullulan, dextran, dextrin, chondroitin sulfate, sodium chondroitin sulfate, a cellulose derivative (for example, a water-soluble cellulose derivative obtained by partially modifying cellulose such as carboxymethyl cellulose, hydroxypropyl cellulose, or hydroxypropyl methylcellulose), hydroxyethyl starch, and gum arabic. The above-described components may be used alone or in the form of a mixture of two or more kinds thereof.

Among these, as the water-soluble polymer contained in needles, at least one selected from the group consisting of hydroxyethyl starch, dextran, chondroitin sulfate, sodium chondroitin sulfate, sodium hyaluronate, carboxymethyl cellulose, polyvinylpyrrolidone, polyoxyethylene polyoxypropylene glycol, polyethylene glycol, and polyvinyl alcohol is preferable, and hydroxyethyl starch is particularly preferable. Further, an electrically neutral water-soluble polymer is preferable because aggregation is unlikely to occur in a case where the polymer is mixed with a drug. The water-soluble polymer contained in the needles may be the same as or different from the water-soluble polymer contained in the sheet.

The weight-average molecular weight of the water-soluble polymer contained in the needles is preferably in a range of 1000 to 300000, more preferably in a range of 3000 to 200000, and still more preferably in a range of 5000 to 100000.

The disaccharides contained in the needles (particularly, needle tips) are not particularly limited, and examples thereof include sucrose, lactulose, lactose, maltose, trehalose, and cellobiose. Among these, sucrose, maltose, and trehalose are preferable.

In the present invention, the content of the water-soluble polymer or disaccharides is 50% by mass or greater with respect to the total solid content of the needle. The content of the water-soluble polymer or disaccharides is preferably 55% by mass or greater, more preferably 60% by mass or greater, and still more preferably 65% by mass or greater with respect to the total solid content of the needle.

The upper limit thereof is not particularly limited, but the content of the water-soluble polymer or disaccharides is set to preferably 99.99% by mass or less with respect to the total solid content of the needle, more preferably 99.9% by mass or less with respect to the total solid content of the needle, and still more preferably 99% by mass or less with respect to the total solid content of the needle.

In a case where the content of the water-soluble polymer or disaccharides is set to 50% by mass or greater with respect to the total solid content of the needle, excellent puncture properties and excellent drug efficacy can be achieved.

The proportion of the water-soluble polymer or disaccharide in the total solid content of the needle can be measured according to the following method, but the present invention is not particularly limited thereto. For example, according to the measuring method, the proportion can be measured by cutting needles of a prepared microneedle array, dissolving the needles in a buffer solution (a buffer solution suitable for dissolving the water-soluble polymer constituting the needles, such as phosphate-buffered saline (PBS)), and measuring the amount of the water-soluble polymer or disaccharides in the solution using a high performance liquid chromatography method.

The needle contains a drug.

The drug is a substance that affects a human body. It is preferable that the drug is selected from peptides (including peptide hormones or the like) or derivatives thereof, proteins, nucleic acids, polysaccharides, vaccines, adjuvants, pharmaceutical compounds belonging to a water-soluble low-molecular-weight compound, and cosmetic ingredients. The molecular weight of the drug is not particularly limited, but a drug having a molecular weight of 500 or greater is preferable in a case of proteins.

Examples of peptides or derivatives thereof and proteins include calcitonin, adrenocorticotropic hormone, parathyroid hormone (PTH), human PTH (1→34), insulin, exendin, secretin, oxytocin, angiotensin, β-endorphin, glucagon, vasopressin, somatostatin, gastrin, luteinizing hormone releasing hormone, enkephalin, neurotensin, atrial natriuretic peptide, growth hormone, growth hormone releasing hormone, bradykinin, substance P, dynorphin, thyroid stimulating hormone, prolactin, interferon, interleukin, granulocyte colony stimulating factor (G-CSF), glutathione peroxidase, superoxide dismutase, desmopressin, somatomedin, endothelin, and salts thereof.

Examples of the vaccines include influenza antigen (influenza vaccine), hepatitis B virus surface antigen (HBs) antigen, hepatitis Be antigen (HBe antigen), Bacille de calmette et Guerin (BCG) antigen, measles antigen, rubella antigen, varicella antigen, yellow fever antigen, shingles antigen, rotavirus antigen, influenza bacilli b type (Hib) antigen, rabies antigen, cholera antigen, diphtheria antigen, pertussis antigen, tetanus antigen, inactivated polio antigen, Japanese encephalitis antigen, human papilloma antigen, and antigens obtained by mixing two to four types of these.

Examples of the adjuvants include aluminum salts such as aluminum phosphate, aluminum chloride, and aluminum hydroxide, emulsions such as MF59 (registered trademark) and AS03 (trade name), liposomes, plant-derived components, nucleic acids, biopolymers, cytokine, peptides, proteins, and sugar chains.

Among these, as the drug, at least one selected from the group consisting of peptide hormones, vaccines, and adjuvants is preferable, and peptide hormones or vaccines are particularly preferable. As the peptide hormones, growth hormone is particularly preferable. As the vaccines, the influenza vaccine is particularly preferable.

The content of the drug in all needles is not particularly limited, but is preferably in a range of 0.001% to 20% by mass, more preferably in a range of 0.003% to 10% by mass, and particularly preferably in a range of 0.005% to 5% by mass with respect to the mass of the solid content of needles.

In the present invention, the mass of the drug in a needle tip region including the needle tip and having a height corresponding to a length of 2/3, 575/800, or 1/2 of a height of the entire needle is preferably 80% or greater, more preferably 85% or greater, and still more preferably 90% or greater of the total mass of the drug that has filled the mold.

The proportion of the mass of the drug in the needle tip region can be measured by the following method, but the present invention is not particularly limited thereto.

For example, according to the measuring method, the proportion can be measured by cutting the sheet and the region including the needle tip, which is the needle tip region having a height corresponding to a length of 2/3, 575/800, or 1/2 of the height of the entire needle and dissolving the cut needle tip region in a buffer solution (a buffer solution suitable for dissolving the drug constituting the needles, such as tris(trishydroxymethylaminomethane) buffer solution). The amount of the drug dissolved in the solution can be measured according to an enzyme-linked immunosorbent assay (ELISA) method or the like.

The needles contain a surfactant.

The surfactant may be any of a nonionic surfactant (an electrically neutral surfactant), a cationic surfactant, an anionic surfactant, or an amphoteric surfactant. Among these, a nonionic surfactant (an electrically neutral surfactant) is preferable.

Examples of the nonionic surfactant include sugar alcohol fatty acid ester such as sucrose fatty acid ester, sorbitan fatty acid esters, glycerin fatty acid ester, polyglycerin fatty acid ester, propylene glycol fatty acid ester, polyoxyethylene sorbitan fatty acid ester, polyoxyethylene glycerin fatty acid ester, polyethylene glycol fatty acid ester, a polyoxyethylene/polyoxypropylene copolymer, polyoxyethylene castor oil, polyoxyethylene hydrogenated castor oil, and octylphenol ethoxylate. Among these, sorbitan fatty acid ester, a polyoxyethylene/polyoxypropylene copolymer, or polyoxyethylene hydrogenated castor oil is particularly preferable. As the nonionic surfactant, commercially available products such as Tween (registered trademark) 80, Pluronic (registered trademark) F-68, HCO-60, and Triton (registered trademark)-X can also be used.

Examples of the cationic surfactant include a quaternary ammonium compound (such as benzalkonium chloride, cetylpyridinium chloride, benzethonium chloride, or cetyltrimethylammonium bromide), and other trimethylalkylammonium salts.

Examples of the anionic surfactant include salts of perfluorinated carboxylic acid and perfluorinated sulfonic acid, an alkyl sulfate (such as sodium dodecyl sulfate or ammonium lauryl sulfate), ether sulfate (such as sodium lauryl ether sulfate), and an alkylbenzene sulfone.

Examples of the amphoteric surfactant include dodecyl betaine, cocoamphoglycinate, and cocamidopropyl betaine.

The amount of the surfactant to be added is not particularly limited, but is preferably 0.01 μg or greater and more preferably 0.05 μg or greater per one microneedle array (an area of 1 cm²).

Hereinafter, preferred embodiments of the present invention will be described with reference to the accompanying drawings, but the present invention is not limited thereto.

FIGS. 1 to 8 are partially enlarged views illustrating a microneedle 110 in the microneedle array. The microneedle array of the present invention is configured by formation of a plurality of needles 112 on the surface of the sheet 116 (in the figures, only one needle 112 is shown on the sheet 116 or one frustum 113 and one needle 112 are shown on the sheet 116 and this is referred to as the microneedle 110).

The needle 112 has a conical shape in FIG. 1A and the needle 112 has a square pyramid shape in FIG. 1B. In FIG. 1C, H represents the height of the needle 112, W represents the diameter (width) of the needle 112, and T represents the height (thickness) of the sheet 116.

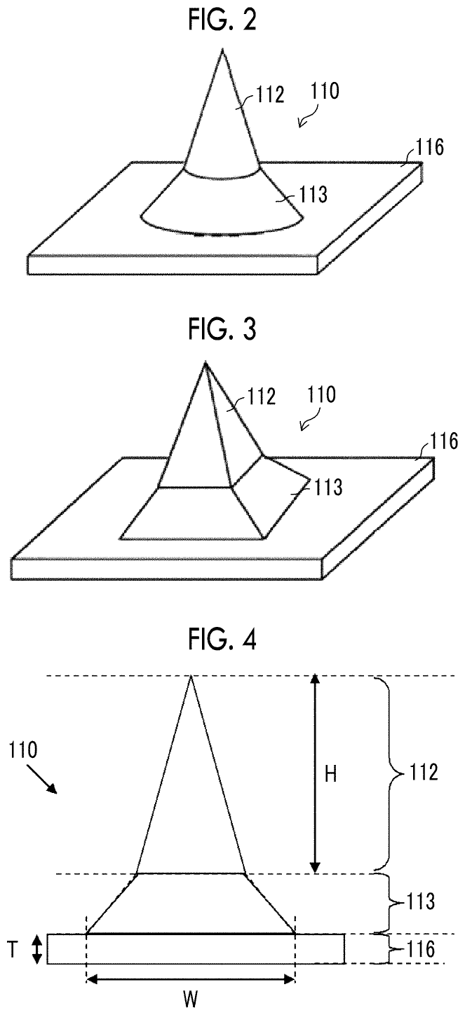

FIGS. 2 and 3 illustrate microneedles 110, on which the frustum 113 and the needle 112 are formed and which have different shapes, formed on the surface of the sheet 116. In FIG. 2, the frustum 113 has a truncated conical shape and the needle 112 has a conical shape. In FIG. 3, the frustum 113 has a truncated square pyramid shape and the needle 112 has a square pyramid shape. However, the shape of the needle is not particularly limited.

FIG. 4 is a cross-sectional view of the microneedles 110 illustrated in FIGS. 2 and 3. In FIG. 4, H represents the height of the needle 112, W represents the diameter (width) of the base portion, and T represents the height (thickness) of the sheet 116.

It is preferable that the microneedle array of the present invention has a shape of the microneedle 110 of FIG. 4 other than the shape of the microneedle 110 in FIG. 1C. With such a configuration, the volume of all needles increases so that a greater amount of drug can be concentrated at the tip of a needle in a case of producing the microneedle array.

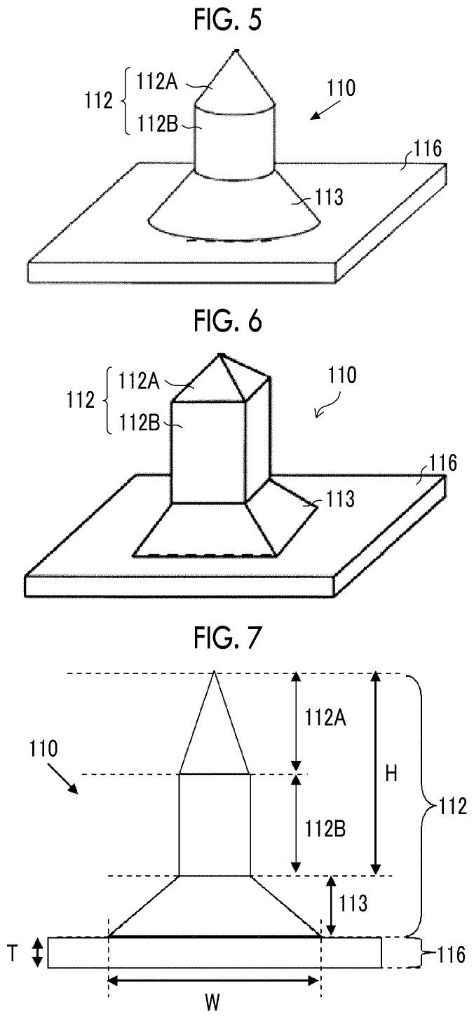

FIGS. 5 and 6 illustrate microneedles 110 in different shapes.

In FIG. 5, a first layer 112A of the needle has a conical shape and a second layer 112B of the needle has a columnar shape. In FIG. 6, the first layer 112A of the needle has a square pyramid shape and the second layer 112B has a square columnar shape. However, the shape of the needle is not particularly limited.

FIG. 7 is a cross-sectional view of the microneedles 110 illustrated in FIGS. 5 and 6. In FIG. 7, H represents the height of the needle 112, W represents the diameter (width) of the base portion, and T represents the height (thickness) of the sheet 116.

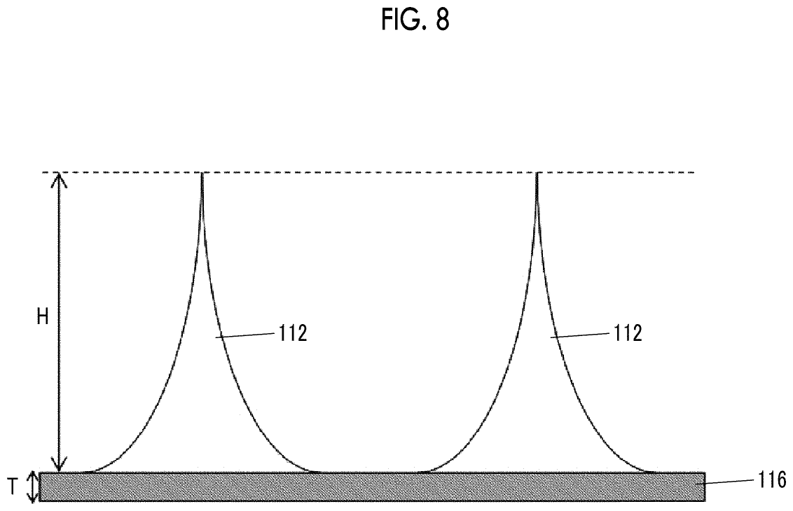

FIG. 8 is a cross-sectional view of a microneedle in another shape in which the inclination (angle) of the side surface of the needle 112 is continuously changed. In FIG. 8, H represents the height of the needle 112 and T represents the height (thickness) of the sheet 116.

In the microneedle array, it is preferable that needles are arranged at intervals of approximately 0.1 to 10 needles per 1 mm in a row. It is more preferable that the microneedle array has 1 to 10000 microneedles per 1 cm². In a case where the density of microneedles is set to 1 needle/cm² or greater, the microneedles can efficiently puncture the skin. Further, in a case where the density of the microneedles is set to 10000 needles/cm² or less, the microneedle array can sufficiently puncture the skin. The density of needles is preferably in a range of 10 to 5000 needles/cm², more preferably in a range of 25 to 1000 needles/cm², and particularly preferably in a range of 25 to 400 needles/cm².

The microneedle array can be supplied in a sealed storage form together with a drying agent. As the drying agent, known drying agents (such as silica gel, calcined lime, calcium chloride, silica alumina, and a sheet-like drying agent) can be used.

[Method of Producing Microneedle Array]

The present invention relates to a method of producing a microneedle array, including a step of filling a hydrophobic mold with a drug-containing solution to form a needle tip, and a step of filling the mold including the formed needle tip with a liquid which contains a water-soluble polymer or disaccharides to form a needle base and a sheet, in which the drug-containing solution contains 0.01 mg/mL to 5 mg/mL of a surfactant.

In the present invention, for example, a microneedle array can be produced according to the following method in conformity with the method described in JP2013-153866A or WO2014/077242A.

(Preparation of Mold)

The mold used in the present invention is a hydrophobic mold. It is preferable that the mold is a mold having a silicon atom or a carbon atom.

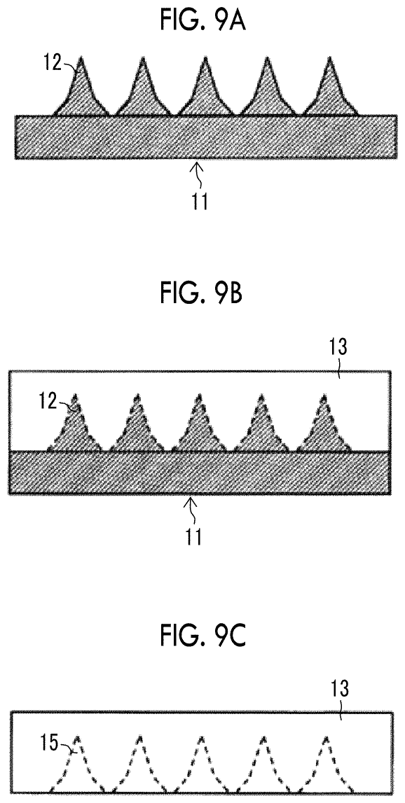

FIGS. 9A to 9C are step views illustrating a method of preparing a mold (die). As illustrated in FIG. 9A, first, an original plate is prepared in order to prepare the mold. There are two methods for preparing an original plate 11.

According to the first method, a Si substrate is coated with a photoresist, exposed, and then developed. Further, an array of shaped portions 12 having a conical shape (projection) is prepared on the surface of the original plate 11 by performing etching using reactive ion etching (RIE) or the like. In a case where the etching such as RIE or the like is performed so as to form shaped portions having a conical shape on the surface of the original plate 11, the portions having a conical shape can be formed by performing etching in an oblique direction while the Si substrate rotates. According to the second method, an array of the shaped portions 12 having a square pyramid shape or the like is formed on the surface of the original plate 11 by performing processing on a metal substrate such as Ni using a cutting tool such as a diamond bit.

Next, a mold is prepared. Specifically, a mold 13 is prepared using the original plate 11 as illustrated in FIG. 9B. As the method of preparing the mold, four methods are considered.

According to the first method, a silicone resin obtained by adding a curing agent to polydimethylsiloxane (PDMS, for example, SYLGARD 184 (registered trademark, manufactured by Dow Corning Toray Co., Ltd.)) is poured into the original plate 11, subjected to a heat treatment at 100° C., cured, and peeled off from the original plate 11. According to the second method, an ultraviolet (UV) cured resin which is cured by being irradiated with ultraviolet rays is poured into the original plate 11, irradiated with ultraviolet rays in a nitrogen atmosphere, and peeled off from the original plate 11. According to the third method, a solution obtained by dissolving a plastic resin such as polystyrene or polymethyl methacrylate (PMMA) in an organic solvent is poured into the original plate 11 coated with a peeling agent, dried so that the organic solvent is volatilized, and cured, and then peeled off from the original plate 11. According to the fourth method, an inverted product is produced using Ni electroforming.

In this manner, the mold 13 formed by needle-like recesses 15, which have an inverted shape of the conical shape or the pyramid shape of the original plate 11, being two-dimensionally arranged is prepared. The mold 13 prepared in the above-described manner is illustrated in FIG. 9C.

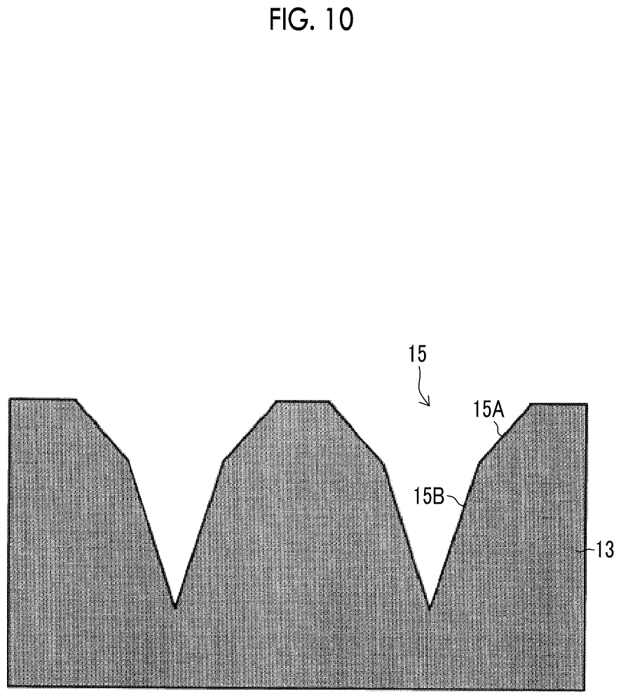

FIG. 10 illustrates another preferred embodiment of the mold 13. The needle-like recess 15 comprises a tapered inlet portion 15A which is narrower in a depth direction from the surface of the mold 13 and a tip recess 15B which becomes tapered in the depth direction. In a case where the inlet portion 15A has a tapered shape, the needle-like recess 15 is easily filled with the solution.

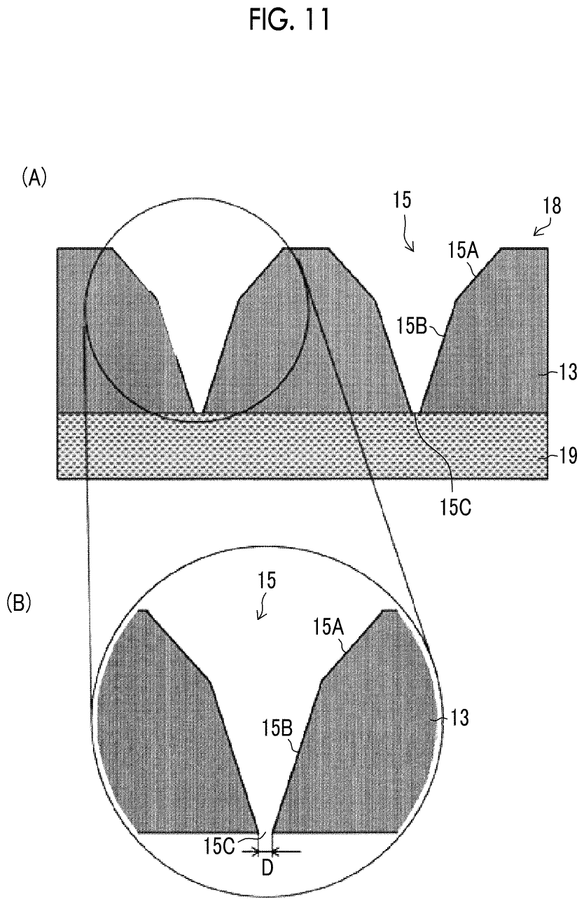

FIG. 11 illustrates a more preferred embodiment of a mold complex 18 in a case of producing the microneedle array. The (A) portion of FIG. 11 illustrates the mold complex 18. The (B) portion of FIG. 11 is a partially enlarged view of a portion enclosed by a circle in the (A) portion.

As illustrated in the (A) portion of FIG. 11, the mold complex 18 comprises the mold 13 having an air vent hole 15C formed on the tip (bottom) of the needle-like recess 15; and an air permeating sheet 19 which is bonded to the rear surface of the mold 13 and is formed of a material that permeates a gas and does not permeate a liquid. The air vent hole 15C is formed as a through-hole penetrating the rear surface of the mold 13. Here, the rear surface of the mold 13 indicates the surface on a side where the air vent hole 15C is formed. With this configuration, the tip of the needle-like recess 15 communicates with the air through the air vent hole 15C and the air permeating sheet 19.

In a case where such a mold complex 18 is used, only the air present in the needle-like recess 15 can be released from the needle-like recess 15 without permeation of the solution filling the needle-like recess 15. In this manner, the property of transferring the shape of the needle-like recess 15 to a polymer is excellent and a sharper needle can be formed.

A diameter D (diameter) of the air vent hole 15C is preferably in a range of 1 to 50 μm. In a case where the diameter D of the air vent hole 15C is less than 1 μm, the air vent hole 15C cannot be sufficiently used as an air bend hole. Further, in a case where the diameter D of the air vent hole 15C is greater than 50 μm, the sharpness of the tip of a formed microneedle is damaged.

As the air permeating sheet 19 formed of a material that permeates a gas and does not permeate a liquid, for example, an air permeating film (POREFLON (registered trademark), FP-010, manufactured by Sumitomo Electric Industries, Ltd.) can be suitably used.

A hydrophobic material may be used as the material used for the mold 13. For example, an elastic material or a metal material can be used. Among these, an elastic material is preferable, and a material having a high gas permeability is more preferable. The oxygen permeability, which is a representative example of the gas permeability, is preferably 1×10⁻¹² (mL/s·m²·Pa) or greater and more preferably 1×10⁻¹⁰ (mL/s·m²·Pa) or greater. Further, 1 mL is 10⁻⁶ m³. In a case where the gas permeability is in the above-described range, the air present in a recess of the mold 13 can be released from the mold and a microneedle array with less defects can be produced. Specific examples of such materials include materials obtained by melting or dissolving, in a solvent, a silicone resin (for example, SYLGARD 184 (registered trademark, manufactured by Dow Corning Toray Co., Ltd.) or KE-1310ST (product number, manufactured by Shin-Etsu chemical Co., Ltd.)), a UV curable resin, or a plastic resin (for example, polystyrene or polymethyl methacrylate (PMMA)). Among these, a silicone rubber-based material is preferable since the material has durability to transfer resulting from repetitive pressure and has excellent peeling properties with respect to a material. Further, examples of the metal material include Ni, Cu, Cr, Mo, W, Ir, Tr, Fe, Co, MgO, Ti, Zr, Hf, V, Nb, Ta, a-aluminum oxide, zirconium oxide, stainless steel (for example, STAVAX (registered trademark) of Bohler-Uddeholm KK), and alloys thereof.

(Solution)

In the present invention, it is preferable to prepare a drug-containing solution (i) (preferably a liquid containing a drug, a surfactant, and at least one of a water-soluble polymer or disaccharides) for forming a needle tip which is a part of a needle, and a liquid (ii) containing a water-soluble polymer or disaccharides for forming a needle base of a needle and a sheet (or a needle base, a frustum, and a sheet).

The kinds of the water-soluble polymer, the disaccharides, the drug, and the surfactant are as described above in the present specification.

The content of the surfactant in the drug-containing solution is in a range of 0.01 mg/mL to 5 mg/mL and preferably in a range of 0.05 mg/mL to 5 mg/mL. In a case where the content of the surfactant is set to 0.01 mg/mL or greater, the drug can be concentrated at the needle tip. Further, in a case where the content of the surfactant is set to 5 mg/mL or less, the occurrence of needle failure can be suppressed.

The concentration of the water-soluble polymer or disaccharides in the solution varies depending on the kind of the material to be used and is preferably in a range of 1% to 50% by mass. Further, the solvent used for dissolution may be a solvent other than water as long as the solvent has volatility, and methyl ethyl ketone (MEK) or an alcohol can be used as the solvent.

(Formation of Needle Tip)

In the present invention, the hydrophobic mold is filled with the drug-containing solution to form a needle tip.

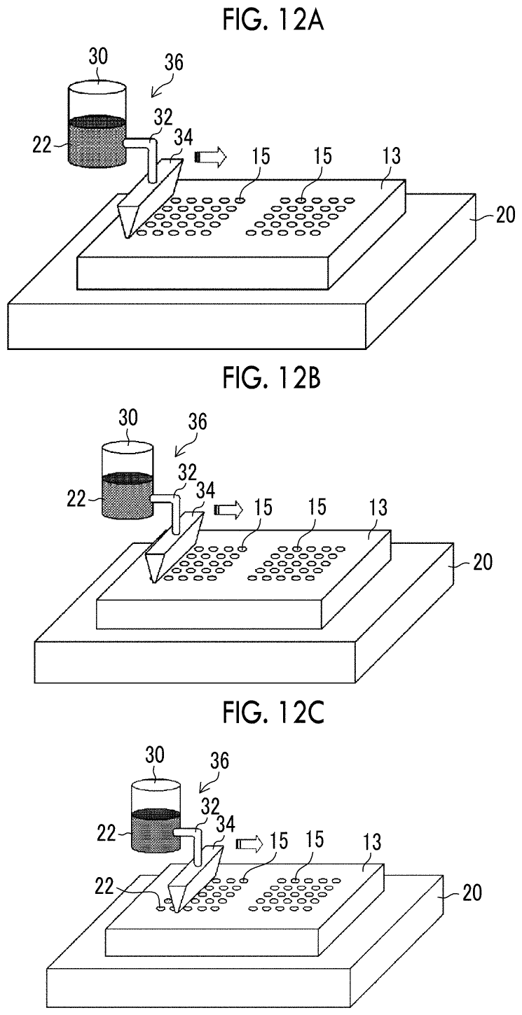

As illustrated in FIG. 12A, the mold 13 having needle-like recesses 15 which are two-dimensionally arranged is disposed on a base 20. In the mold 13, two sets of plural needle-like recesses 15 are formed such that 5 rows of needle-like recesses 15 and 5 columns of needle-like recesses 15 are two-dimensionally arranged. A liquid supply device 36 including a tank 30 which accommodates a drug-containing solution 22, a pipe 32 which is connected with the tank, and a nozzle 34 which is connected with the tip of the pipe 32 is prepared. Further, in the present example, the case where 5 rows of needle-like recesses 15 and 5 columns of needle-like recesses 15 are two-dimensionally arranged is exemplified, but the number of the needle-like recesses 15 is not limited to 5 rows×5 columns as long as the needle-like recesses are two-dimensionally arranged in a manner of M×N (M and N each independently represent an optional integer of 1 or greater, preferably in a range of 2 to 30, more preferably in a range of 3 to 25, and still more preferably in a range of 3 to 20).

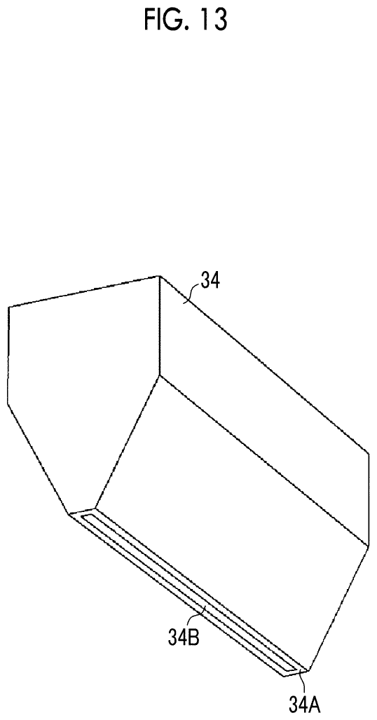

FIG. 13 is a perspective view schematically illustrating the end of the nozzle. As illustrated in FIG. 13, the end of the nozzle 34 comprises a lip portion 34A which is a flat surface and an opening portion 34B having a slit shape. For example, a plurality of needle-like recesses 15 forming one row can be concurrently filled with the drug-containing solution 22 due to the opening portion 34B having a slit shape. The size (the length and the width) of the opening portion 34B is appropriately selected according to the number of needle-like recesses 15 to be concurrently filled with the solution. In a case where the length of the opening portion 34B is increased, more needle-like recesses 15 can be concurrently filled with the drug-containing solution 22. In this manner, the productivity can be improved.

As the material used for the nozzle 34, an elastic material or a metal material can be used. Examples thereof include TEFLON (registered trademark), stainless steel (steel use stainless (SUS)), and titanium.

As illustrated in FIG. 12B, the position of the opening portion 34B of the nozzle 34 is adjusted on the needle-like recesses 15. The lip portion 34A of the nozzle 34 is in contact with the surface of the mold 13. The drug-containing solution 22 is supplied to the mold 13 from the liquid supply device 36, and the needle-like recesses 15 are filled with the drug-containing solution 22 from the opening portion 34B of the nozzle 34. In the present embodiment, the plurality of needle-like recesses 15 constituting one row are concurrently filled with the drug-containing solution 22. However, the present invention is not limited thereto, and the needle-like recesses 15 can be filled with the solution one by one.

In a case where the mold 13 is formed of a material having a gas permeability, the drug-containing solution 22 can be sucked from the rear surface of the mold 13, and the filling of the needle-like recesses 15 with the drug-containing solution 22 can be promoted.

Next to the filling step of FIG. 12B, as illustrated in FIG. 12C, the liquid supply device 36 is relatively moved in a direction perpendicular to the length direction of the opening portion 34B while the lip portion 34A of the nozzle 34 is brought into contact with the surface of the mold 13, and the nozzle 34 is moved to the needle-like recesses 15 which are not filled with the drug-containing solution 22. The position of the opening portion 34B of the nozzle 34 is adjusted on the needle-like recesses 15. In the present embodiment, the example of moving the nozzle 34 has been described, but the mold 13 may be moved.

Since the movement is made while the lip portion 34A of the nozzle 34 is brought into contact with the surface of the mold 13, the drug-containing solution 22 remaining on the surface of the mold 13 other than the needle-like recesses 15 can be collected by the nozzle 34. It is possible to prevent the drug-containing solution 22 from remaining in portions other than the needle-like recesses 15 of the mold 13.

In order to reduce the damage to the mold 13 and suppress deformation due to compression of the mold 13 as much as possible, it is preferable that the pressing pressure of the nozzle 34 against the mold 13 is set to be as small as possible during the movement. Further, in order to prevent the drug-containing solution 22 from remaining on the surface of the mold 13 other than the needle-like recesses 15, it is desirable that at least one of the mold 13 or the nozzle 34 is formed of a flexible material which can be elastically deformed.

By repeating the filling step of FIG. 12B and the moving step of FIG. 12C, 5 rows and 5 columns of needle-like recesses 15 which are two-dimensionally arranged are filled with the drug-containing solution 22. In a case where 5 rows and 5 columns of needle-like recesses 15 which are two-dimensionally arranged are filled with the drug-containing solution 22, the liquid supply device 36 is moved to 5 rows and 5 columns of adjacent needle-like recesses 15 which are two-dimensionally arranged, and the filling step of FIG. 12B and the moving step of FIG. 12C are repeated. Further, 5 rows and 5 columns of adjacent needle-like recesses 15 which are two-dimensionally arranged are also filled with the drug-containing solution 22.

The filling step and the moving step described above may be carried out by (1) filling the needle-like recesses 15 with the drug-containing solution 22 while moving the nozzle 34 or by (2) temporarily stopping the nozzle 34 on the needle-like recesses 15 during the movement of the nozzle 34 to fill the needle-like recesses 15 with the drug-containing solution 22 and moving the nozzle 34 again after the filling. The lip portion 34A of the nozzle 34 is brought into contact with the surface of the mold 13 between the filling step and the moving step.

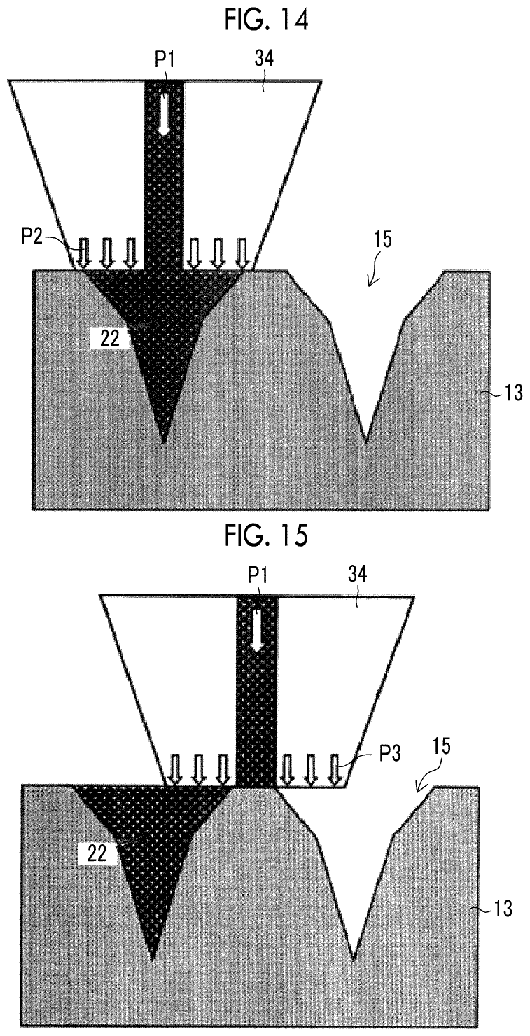

FIG. 14 is a partially enlarged view illustrating the end of the nozzle 34 and the mold 13 while the needle-like recesses 15 are filled with the drug-containing solution 22. As illustrated in FIG. 14, the filling of the needle-like recesses 15 with the drug-containing solution 22 can be promoted by applying a pressing pressure P1 into the nozzle 34. Further, in a case where the needle-like recesses 15 are filled with the drug-containing solution 22, it is preferable that a pressing force P2 for bringing the nozzle 34 into contact with the surface of the mold 13 is set to be greater than or equal to the pressing pressure P1 applied into the nozzle 34. In a case where the pressing force P2 is set to be greater than or equal to the pressing pressure P1, it is possible to suppress leaking of the drug-containing solution 22 to the surface of the mold 13 from the needle-like recesses 15.

FIG. 15 is a partially enlarged view of the tip of the nozzle 34 and the mold 13 during the movement of the nozzle 34. In a case where the nozzle 34 is relatively moved with respect to the mold 13, it is preferable that a pressing force P3 of bringing the nozzle 34 into contact with the surface of the mold 13 is set to be smaller than the pressing force P2 of bringing the nozzle 34 into contact with the surface of the mold 13 during the filling. The pressing force P3 is set to be smaller than the pressing force P2 in order to reduce the damage to the mold 13 and suppress the deformation of the mold 13 due to compression.

In a case where the filling of the plurality of needle-like recesses 15 which are 5 rows and 5 columns of needle-like recesses is completed, the nozzle 34 is moved to the plurality of adjacent needle-like recesses 15 which are 5 rows and 5 columns of needle-like recesses. In regard to the liquid supply, it is preferable that the supply of the drug-containing solution 22 is stopped in a case where the nozzle 34 is moved to the plurality of adjacent needle-like recesses 15 which are 5 rows and 5 columns of needle-like recesses. There is a distance between the needle-like recesses 15 in the fifth row and the needle-like recesses 15 in the next first row. In a case where the drug-containing solution 22 is continuously supplied while the nozzle 34 is moved between the needle-like recess in the fifth row and the needle-like recess in the next first row, the liquid pressure in the nozzle 34 may be extremely increased. As a result, the drug-containing solution 22 may flow out of the needle-like recesses 15 of the mold 13 from the nozzle 34. In order to suppress this flowing out, it is preferable that the supply of the drug-containing solution 22 is stopped in a case where the liquid pressure in the nozzle 34 is detected and the liquid pressure is determined to be extremely high.

Hereinbefore, the method of supplying the drug-containing solution using a dispenser that has a nozzle has been described, but bar coating, spin coating, or spray coating can be applied in addition to the coating with the dispenser.

In the present invention, it is preferable to perform a drying treatment after the drug-containing solution is supplied to the needle-like recesses.

It is preferable that the microneedle array can be produced by performing a step of drying a mold for forming a needle which has been filled with a drug-containing solution to form a needle tip; and a step of filling the upper surface of the needle tip formed in the above-described manner with the liquid containing a water-soluble polymer or disaccharides and drying the liquid.

It is preferable that the condition for drying the mold for forming a needle which has been filled with the drug-containing solution is that the moisture content of the solution reaches 20% or less after 30 minutes to 300 minutes from the start of the drying.

It is particularly preferable that the drying can be controlled such that the temperature is held to be lower than or equal to a temperature at which the drug does not lose the effect and the moisture content of the solution reaches 20% or less after 60 minutes or longer from the start of the drying.

As a method of controlling the drying rate, any method of delaying the drying, such as the temperature, the humidity, the dry air volume, the use of a container, and the volume and/or the shape of a container, can be employed.

It is preferable that the drying can be performed in a state where the mold for forming a needle which has been filled with the drug-containing solution is covered with a container or stored in a container.

The temperature during the drying is preferably in a range of 1° C. to 45° C. and more preferably in a range of 1° C. to 40° C.

The relative humidity during the drying is preferably in a range of 10% to 95%, more preferably in a range of 20% to 95%, and still more preferably in a range of 30% to 95%.

(Formation of Needle Base and Sheet)

In the present invention, the needle base and the sheet are formed by filling the mold that includes the needle tip which has been formed in the above-described manner with a liquid containing a water-soluble polymer or disaccharides.

Several embodiments of a step of forming the needle base and the sheet will be described.

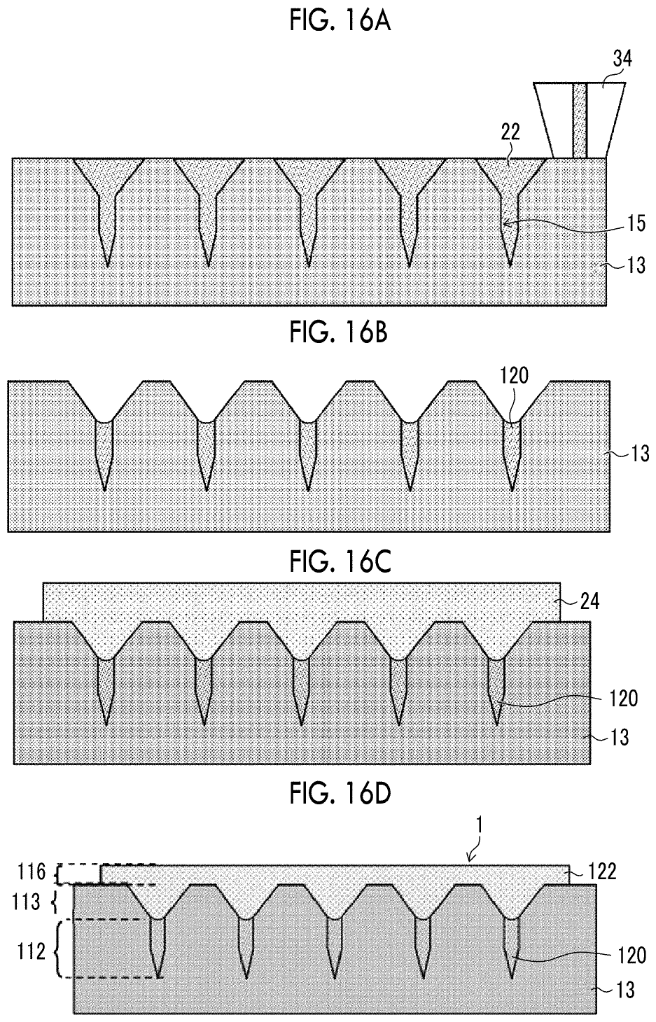

A first embodiment of a step of forming the sheet will be described with reference to FIGS. 16A to 16D. The needle-like recesses 15 of the mold 13 are filled with the drug-containing solution 22 from the nozzle 34. Next, as illustrated in FIG. 16B, the drug-containing solution 22 is dried and solidified to form a layer 120 containing a drug in the needle-like recesses 15. Subsequently, the mold 13 on which the layer 120 containing a drug has been formed is coated with a liquid 24 containing a water-soluble polymer or disaccharides using a dispenser as illustrated in FIG. 16C. In addition to the coating using a dispenser, bar coating, spin coating, or spray coating can be applied. Since the layer 120 containing a drug is solidified, it is possible to suppress the diffusion of the drug in the liquid 24. Next, the microneedle array 1 including the plurality of needles 112, the frustums 113, and the sheet 116 is formed by drying and solidifying the liquid 24 as illustrated in FIG. 16D.

In the first embodiment, in order to promote the filling of the needle-like recesses 15 with the drug-containing solution 22 and the liquid 24 containing a water-soluble polymer or disaccharides, it is preferable to apply a pressure from the surface of the mold 13 and perform suctioning from the rear surface of the mold 13 under reduced pressure.

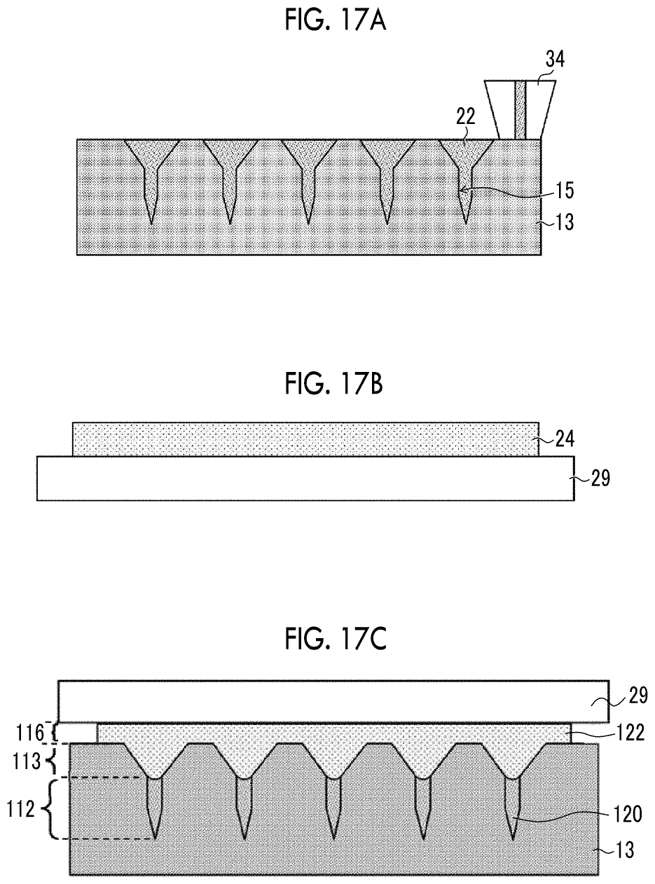

Next, a second embodiment will be described with reference to FIGS. 17A to 17C. As illustrated in FIG. 17A, the needle-like recesses 15 of the mold 13 are filled with the drug-containing solution 22 from the nozzle 34. Next, similarly to FIG. 16B, the layer 120 containing a drug is formed in the needle-like recesses 15 by drying and solidifying the drug-containing solution 22. Next, another support 29 is coated with the liquid 24 containing a water-soluble polymer or disaccharides as illustrated in FIG. 17B. The support 29 is not limited, and examples of the support include polyethylene, polyethylene terephthalate, polycarbonate, polypropylene, an acrylic resin, triacetyl cellulose, and glass. Subsequently, the liquid 24 formed on the support 29 overlaps with the mold 13 having the layer 120 containing a drug formed on the needle-like recesses 15 as illustrated in FIG. 17C. In this manner, the needle-like recesses 15 are filled with the liquid 24. Since the layer containing a drug is solidified, it is possible to suppress the diffusion of the drug in the liquid 24. Next, a microneedle array formed of the plurality of needles 112, the frustums 113, and the sheet 116 is formed by drying and solidifying the liquid 24.

In the second embodiment, in order to promote the filling of the needle-like recesses 15 with the liquid 24 containing a water-soluble polymer or disaccharides, it is preferable to apply a pressure from the surface of the mold 13 and perform suctioning from the rear surface of the mold 13 under reduced pressure.

As the method of drying the liquid 24 containing a water-soluble polymer or disaccharides, a step of volatilizing the solvent in the solution may be used. The method is not particularly limited, and a method of performing heating, blowing air, or decompression may be used. The drying treatment can be performed under the conditions of 1° C. to 50° C. for 1 to 72 hours. Examples of the method of blowing air include a method of blowing hot air at 0.1 to 10 msec. It is preferable that the drying temperature is a temperature at which the drug in the drug-containing solution 22 is not thermally degraded.

(Peeling)

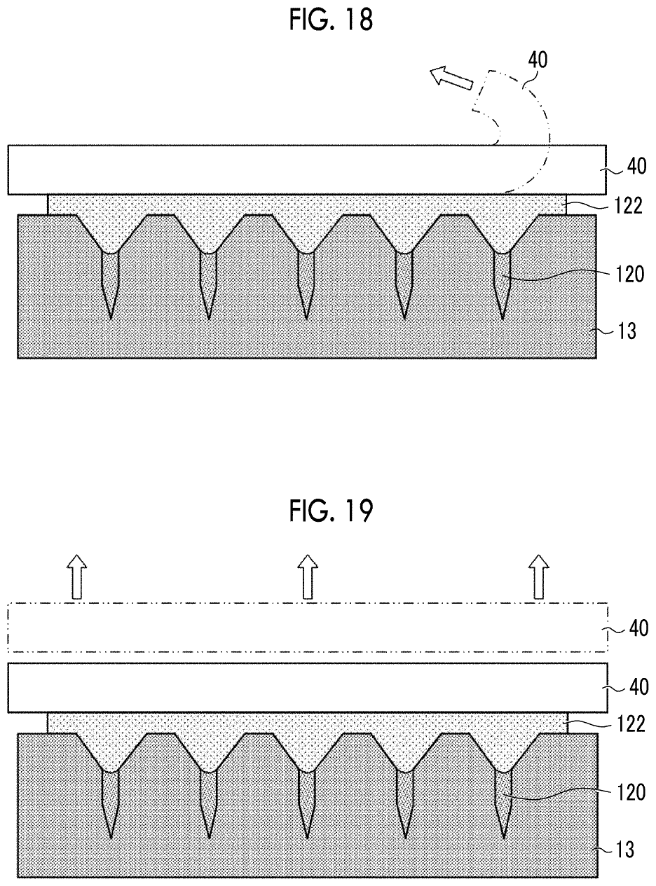

A method of peeling the microneedle array from the mold 13 is not particularly limited. It is preferable that needles are not bent or broken during the peeling. Specifically, a sheet-like base material 40 on which a pressure sensitive adhesive layer is formed is attached to the microneedle array and then the base material 40 can be peeled off from the end portion such that the base material 40 is turned over as illustrated in FIG. 18. However, the needle projections can be bent in a case of using this method. Therefore, as illustrated in FIG. 19, a method of disposing a sucking disc (not illustrated) on the base material 40 provided on the microneedle array and vertically pulling the base material up while suctioning the base material with air can be applied. Further, the support 29 may be used as the base material 40.

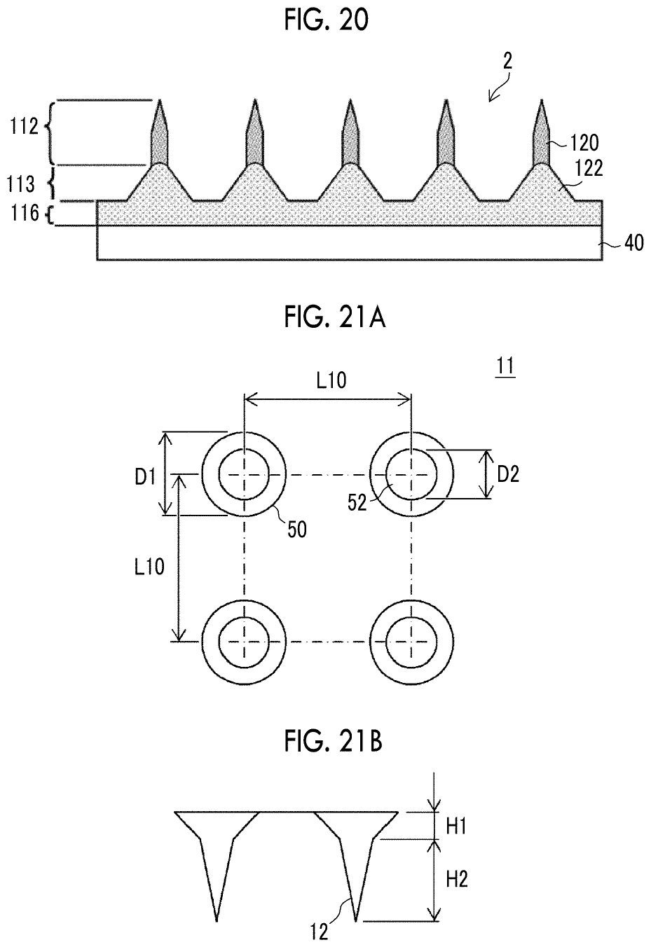

FIG. 20 illustrates the microneedle array 2 peeled off from the mold 13. The microneedle array 2 includes the base material 40, the needles 112 formed on the base material 40, the frustums 113, and the sheet 116. At least the tip of the needle 112 has a conical shape or a polygonal pyramid shape, but the shape of the needle 112 is not limited thereto.

The method for producing the microneedle array according to the embodiment of the present invention is not particularly limited, but it is preferable that the microneedle array is obtained by a production method including a step (1) of producing a mold, a step (2) of preparing a liquid containing a drug, a surfactant, and at least one of a water-soluble polymer and disaccharides, a step (3) of filling the mold with the liquid obtained in the step (2) to form a needle tip region, a step (4) of filling the mold with a liquid containing a water-soluble polymer or disaccharides to form the remaining part of the needle (the frustum as desired) and a sheet, and a step (5) of peeling the microneedle array from the mold.

Hereinafter, the present invention will be described in detail with reference to examples. The materials, the amounts of the materials to be used, the ratios, the treatment contents, and the treatment procedures shown in the examples described below can be appropriately changed as long as they are within the gist of the present invention. Accordingly, the scope of the present invention should not be limitatively interpreted by the specific examples described below.

### EXAMPLES

The abbreviations and the trade names in the examples are as follows.

HES: Hydroxyethyl starch 70000 (Fresenius Kabi AG) (weight-average molecular weight of 70000)

CS: Sodium chondroitin sulfate (Maruha Nichiro Corporation) (weight-average molecular weight of 90000)

Sucrose: Sucrose (Wako Pure Chemical Industries, Ltd.)

Tw: Tween (registered trademark) 80 (Seppic)

SDS: Sodium dodecyl sulfate (Wako Pure Chemical Industries, Ltd.)

Pluronic (registered trademark) F-68 (NOF Corporation)

Triton (registered trademark)-X (Alfa Aesar)

<Production of Mold>

An original plate 11 was prepared by arranging shaped portions 12 having a needle-like structure, on which a cone 52 with a diameter D2 of 300 μm and a height H2 of 500 μm was formed, as illustrated in FIG. 21, on a truncated cone 50 having a bottom surface with a diameter D1 of 500 μm and having a height H1 of 150 μm, on the surface of a smooth Ni plate in which each side had a length of 40 mm and performing grinding processing on 100 needles having a pitch L10 of 1000 μm and a square pyramid shape in a two-dimensional square array. A film of silicon rubber (SILASTIC MDX 4-4210, manufactured by Dow Corning Toray Co., Ltd.) was formed on the original plate 11 such that the thickness thereof reached 0.6 mm, and the film was thermally cured in a state where 50 μm of the conical tip of the original plate 11 protruded from the film surface and then peeled off. In this manner, an inverted product of the silicon rubber having a through-hole with a diameter of approximately 30 μm was prepared. The silicon rubber inverted product which had 10 rows and 10 columns of needle-like recesses that were two-dimensionally arranged and formed on the central portion and in which the portion other than the flat surface portion in which each side had a length of 30 mm was cut off was used as a mold. A surface on which the opening portion of a needle-like recess was wide was set to the front surface of the mold and a surface having a through-hole (air vent hole) with a diameter of 30 μm was set to the rear surface of the mold.

<Experiment Using Human Serum Albumin>

(Preparation of Aqueous Solution Containing Human Serum Albumin that Forms Needle Tip)

Lyophilized human serum albumin (Wako Pure Chemical Industries, Ltd.) was dissolved in water for injection and mixed with hydroxyethyl starch 70000 (HES, Fresenius Kabi AG, weight-average molecular weight of 70000), sucrose (Wako Pure Chemical Industries, Ltd.), and Tween (Registered trademark) 80 (Seppic) (in a case of examples). The amounts of the human serum albumin, HES, sucrose, and surfactant (Tween (registered trademark) 80) were adjusted as listed in Table 1.

(Preparation of Water-Soluble Polymer-Dissolved Solution that Forms Needle Base and Sheet)

Sodium chondroitin sulfate (CS, Maruha Nichiro Co., Ltd.) was dissolved in water to prepare a 39 mass % aqueous solution of CS.

(Filling of Mold with Aqueous Solution Containing Human Serum Albumin)

The mold produced in the above-described manner was filled with the aqueous solution containing human serum albumin which had been prepared in the above-described manner. Subsequently, the mold was placed in a lid (box) in an environment of 23° C. and a relative humidity of 45% and dried. At this time, the aqueous solution containing human serum albumin was gradually dried, and the moisture content thereof reached 20% or less after 180 minutes or longer. The method of drying the solution is not limited to using a lid, and other methods of controlling the temperature and the humidity or controlling the air volume may be used.

(Forming and Drying of Needle Base and Sheet)

As a support for forming the sheet, a support obtained by performing a hydrophilized plasma treatment on a polyethylene terephthalate (PET) sheet (175 μm) using a cloud remover (manufactured by Victor Company of Japan, Ltd.) under the following conditions (used gas: O₂, gas pressure: 13 Pa, radio frequency (RF) power: 100 W, irradiation time: 3 minutes, O₂ flow rate: SV250, and target degree of vacuum (CCG): 2.0×10⁻⁴ Pa). The treated PET was coated with the water-soluble polymer-dissolved solution such that the film thicknesses of the front and rear surfaces thereof reached 75 μm. Meanwhile, the mold filled with the polymer-dissolved solution containing a drug was suctioned and fixed to a suction table. The surface of the PET coated with the water-soluble polymer-dissolved solution was disposed to face the surface of the mold, and a space between the PET and the mold and a space on a side of the PET opposite to the mold were decompressed for 2 minutes. After the decompression, the mold was bonded to the PET coated with the water-soluble polymer-dissolved solution by releasing the atmospheric pressure only in the space of the PET opposite to the mold. After the contact state was maintained for 10 minutes, the integrated body obtained by bonding the PET and the mold was dried in an environment of 23° C. and 45 RH % (relative humidity %).

(Peeling Step)

The dried and solidified microneedle array was carefully peeled off from the mold to form a microneedle array containing human serum albumin.

The microneedle is formed of a frustum and a needle and has a truncated cone structure in which the needle-like projection has a length L of approximately 600 μm in height and has a base portion with a width of approximately 270 μm, and the frustum has a height of approximately 140 μm and has an upper bottom with a diameter of approximately 270 μm and a lower bottom with a diameter of 500 μm, and the number of needles is 100 and the needles are arranged at intervals of 1 mm.

(Evaluation 1: Confirmation of Microneedle Array Shape)

The prepared microneedle array was observed with a microscope (VHX-5000, Keyence Corporation). The observation results are listed in Table 1. In Table 1, the microneedle array whose shape was observed to be abnormal (cracks or cloudiness) was evaluated as B, and the microneedle array whose shape was not observed to be abnormal was evaluated as A.

(Evaluation 2: Evaluation of Filling Rate of Needle Tip)

The needle tip region of the prepared microneedle array covering from the needle tip to approximately 400 μm was cut in parallel with the sheet, and the cut needle tip region was dissolved in pure water (sample 1). Similarly, the microneedle array remaining after the cutting was dissolved in pure water (sample 2). The amount of the human serum albumin in the solution was measured according to the Bradford method. The filling rate of the needle tip was acquired based on the following equation. A case where the filling rate of the needle tip was 80% or greater was evaluated as A, and a case where the filling rate of the needle tip was less than 80% was evaluated as B.

Filling rate of needle tip=amount of vaccine in sample 1÷ (amount of vaccine in sample 1+amount of vaccine in sample 2)

TABLE 1

| | | | | | | | Result | |
|---|---|---|---|---|---|---|---|---|
| | Aqueous solution containing human serum albumin | | | Water-soluble | | | Filling rate | |
| | | Base | | polymer-dissolved | Needle | | of needle tip | Needle |
| | Drug | material | Surfactant | solution | shape | Support | (400 μm from tip) | shape |
| Comparative | Human serum | HES | None | CS 39% | 600 μm | PET support | B (70%) | A |
| Example 1 | albumin | 166 mg/m | | | 100 needles | is available | | |
| | 66 mg/mL | Sucrose | | | | | | |
| | | 26 mg/mL | | | | | | |
| Example 1 | Human serum | HES | Tween | CS 39% | 600 μm | PET support | A (80%) | A |
| | albumin | 166 mg/mL | (registered trademark) | | 100 needles | is available | | |
| | 66 mg/mL | Sucrose | 80 0.16 mg/mL | | | | | |
| | | 26 mg/mL | | | | | | |

<Experiment Using Influenza Vaccine>

(Preparation of Aqueous Solution Containing Influenza Vaccine that Forms Needle Tip)

The influenza vaccine was concentrated by centrifugation and then mixed with a base and a surfactant (in a case of examples) listed in Table 2. The kinds and amounts of the influenza vaccine, the base, and the surfactant were adjusted as shown in Table 2.

(Preparation of Water-Soluble Polymer-Dissolved Solution that Forms Needle Base and Sheet)

Sodium chondroitin sulfate (CS, Maruha Nichiro Co., Ltd., weight-average molecular weight: 90000) was dissolved in water to prepare a 39 mass % aqueous solution of CS.

(Filling of Mold with Aqueous Solution Containing Influenza Vaccine)

The mold produced in the above-described manner was filled with the aqueous solution containing the influenza vaccine which had been prepared in the above-described manner. Subsequently, the mold was placed in a lid (box) in an environment of 23° C. and a relative humidity of 45% and dried. At this time, the aqueous solution containing the influenza vaccine was gradually dried, and the moisture content thereof reached 20% or less after 180 minutes or longer. The method of drying the solution is not limited to using a lid, and other methods of controlling the temperature and the humidity or controlling the air volume may be used.

(Forming and Drying of Needle Base and Sheet)

A stainless steel (SUS304) frame (with a thickness of 0.2 mm and a diameter of 15 mm) was placed on the mold filled with the aqueous solution. Here, the 39 mass % aqueous solution of CS was directly applied thereto, the needle-like recesses were filled with the solution, and the solution was dried (at a temperature of 23° C. and a relative humidity of 45%).

(Peeling Step)

The dried and solidified microneedle array was carefully peeled off from the mold to form a microneedle array containing the influenza vaccine. The microneedle is formed of a frustum and a needle and has a truncated cone structure in which the needle-like projection has a length L of approximately 800 μm in height and has a base portion with a width of approximately 360 μm, and the frustum has a height of approximately 170 μm and has an upper bottom with a diameter of approximately 360 μm and a lower bottom with a diameter of 750 μm, and the number of needles is 109 and the needles are arranged at intervals of 1 mm.

(Evaluation 1: Confirmation of Microneedle Array (MNA) Shape)

The prepared microneedle array was observed with a microscope (VHX-5000, Keyence Corporation). The observation results are listed in Table 2. In Table 2, the microneedle array whose shape was observed to be abnormal (cracks or cloudiness) was evaluated as B, and the microneedle array whose shape was not observed to be abnormal was evaluated as A.

(Evaluation 2: Evaluation of Filling Rate of Needle Tip)

The needle tip region of the prepared microneedle array covering from the needle tip to approximately 575 μm was cut in parallel with the sheet, and the cut needle tip region was dissolved in a buffer solution (Tween (registered trademark) 20 (Merck), tris buffer solution containing bovine serum albumin (SIGMA)) (sample 1). Similarly, the microneedle array remaining after the cutting was also dissolved in the Buffer solution (sample 2). The amount of the vaccine in the solution was measured according to the enzyme-linked immunosorbent assay (ELISA) method. The filling rate of the needle tip was acquired based on the following equation. A case where the filling rate of the needle tip was 80% or greater was evaluated as A, and a case where the filling rate of the needle tip was less than 80% was evaluated as B.

Filling rate of needle tip=amount of vaccine in sample 1÷ (amount of vaccine in sample 1+amount of vaccine in sample 2)

TABLE 2

| | | | | | | Result | |
|---|---|---|---|---|---|---|---|
| | | | | | | | Filling rate |
| | Aqueous solution containing influenza vaccine | | | Water-soluble | | | of needle tip |
| | | Base | | polymer-dissolved | MNA | Needle | (400 μm from |
| | Drug | material | Surfactant | solution | shape | shape | needle tip) |
| Comparative | Influenza vaccine | HES 25 mg/mL | None | CS 39% | 800 μm | A | B (70%) |
| Example 2 | (8.5 mm/mL as | Arginine 25 mg/mL | | | 109 needles | | |
| | hemagglutinin) | | | | | | |
| Example 2 | | | Pluronic | | | A | A (98%) |
| | | | (registered trademark) | | | | |
| | | | F-68 1 mg/mL | | | | |
| Example 3 | | | Pluronic | | | A | A (98%) |
| | | | (registered trademark) | | | | |
| | | | F-68 5 mg/mL | | | | |
| Example 4 | | | Triton | | | A | A (90%) |
| | | | (registered trademark)-X | | | | |
| | | | 1 mg/mL | | | | |
| Example 5 | | | Tween | | | A | A (90%) |
| | | | (registered trademark) | | | | |
| | | | 80 5 mg/mL | | | | |
| Example 6 | | | Pluronic | | | A | A (85%) |
| | | | (registered trademark) | | | | |
| | | | F-68 0.05 mg/mL | | | | |
| Comparative | Influenza vaccine | HES | None | CS 39% | 800 μm | A | B (50%) |
| Example 3 | (8.5 mm/mL as | 84 mg/mL | | | 109 needles | | |
| | hemagglutinin) | | | | | | |
| Example 7 | | | Pluronic | | | A | A (95%) |
| | | | (registered trademark) | | | | |
| | | | F-68 0.25 mg/mL | | | | |
| Example 8 | | | Pluronic | | | A | A (91%) |
| | | | (registered trademark) | | | | |
| | | | F-68 0.10 mg/mL | | | | |
| Comparative | | Sucrose | None | | | A | B (50%) |
| Example 4 | | 84 mg/mL | | | | | |
| Example 9 | | | Pluronic | | | A | A (98%) |
| | | | (registered trademark) | | | | |
| | | | F-68 5 mg/mL | | | | |
| Comparative | | | Pluronic | | | B | Evaluation was |
| Example 5 | | | (registered trademark) | | | | not made |
| | | | F-68 50 mg/mL | | | | |

### EXPLANATION OF REFERENCES

1: microneedle array
2: microneedle array
110: microneedle
112: needle
112A: first layer of needle
112B: second layer of needle
113: frustum
116: sheet
120: layer containing drug
122: layer which does not contain drug
W: diameter (width)
H: height
T: height (thickness)
11: original plate
12: shaped portion
13: mold
15: needle-like recess
15A: inlet portion
15B: tip recess
15C: air vent hole
D: diameter (diameter)
18: mold complex
19: air permeating sheet
20: base
22 drug-containing solution
24 Liquid containing water-soluble polymer or disaccharide
29: support
30: tank
32: pipe
34: nozzle
34A: lip portion
34B: opening portion
36: liquid supply device
P1: pressing pressure
P2: pressing force
P3: pressing force
40: base material
50: truncated cone
52: cone
D1: diameter
D2: diameter
L10: pitch
H1: height
H2: height

What is claimed is:

1. A method of producing a microneedle array, comprising:
a step of filling a hydrophobic mold with a drug-containing solution to form a needle tip; and
a step of filling the mold including the formed needle tip with a liquid which contains a water-soluble polymer or disaccharides to form a needle base and a sheet,
wherein the drug-containing solution contains 0.01 mg/mL to 5 mg/mL of a surfactant.

2. The method according to claim 1,
wherein the surfactant is a nonionic surfactant.

3. The method according to claim 1,
wherein a mass of the drug in a needle tip region including the needle tip and having a height corresponding to a length of 2/3 or 575/800 of a height of an entire needle is 80% or greater of a total mass of the drug that has filled the mold.

4. The method according to claim 2,
wherein a mass of the drug in a needle tip region including the needle tip and having a height corresponding to a length of 2/3 or 575/800 of a height of an entire needle is 80% or greater of a total mass of the drug that has filled the mold.

5. The method according to claim 1,
wherein the drug includes a peptide or a vaccine.

6. The method according to claim 2,
wherein the drug includes a peptide or a vaccine.

7. The method according to claim 3,
wherein the drug includes a peptide or a vaccine.

8. The method according to claim 4,
wherein the drug includes a peptide or a vaccine.

9. The method according to claim 1,
wherein the mold has a silicon atom or a carbon atom.

10. The method according to claim 2,
wherein the mold has a silicon atom or a carbon atom.

11. The method according to claim 3,
wherein the mold has a silicon atom or a carbon atom.

12. The method according to claim 5,
wherein the mold has a silicon atom or a carbon atom.
